(19) United States
(12) Patent Application Publication
(10) Pub. No.: US 2023/0146223 A1
(43) Pub. Date: May 11, 2023

(54) NUCLEIC ACIDS EXTRACTION SYSTEM AND METHOD BASED ON 3D-PRINTED MICRODEVICE

(71) Applicant: DALIAN UNIVERSITY OF TECHNOLOGY, Dalian, Liaoning (CN)

(72) Inventors: Xiaobin JIANG, Dalian, Liaoning (CN); Peipei LI, Dalian, Liaoning (CN); Menghang LI, Dalian, Liaoning (CN); Zhijie YUAN, Dalian, Liaoning (CN); Gaohong HE, Dalian, Liaoning (CN); Wu XIAO, Dalian, Liaoning (CN); Xiangcun LI, Dalian, Liaoning (CN); Xuemei WU, Dalian, Liaoning (CN)

(21) Appl. No.: 17/914,974

(22) PCT Filed: Jun. 18, 2021
(86) PCT No.: PCT/CN2021/100752

§ 371 Date: Sep. 27, 2022

(30) Foreign Application Priority Data
Jun. 23, 2020 (CN) 202010585511.X

(51) Int. Cl. C12N 15/10; B33Y 80/00

(52) U.S. Cl. CPC C12N 15/1006; B33Y 80/00; B29C 64/124

(57) ABSTRACT

The present invention discloses a nucleic acids extraction system and method based on a 3D-printed microdevice, and belongs to the technical field of nucleic acids extraction. The nucleic acids extraction system is a monomer 3D-printed microdevice or a 3D-printed microdevice prepared by 3D printing technologies, the monomer 3D-printed microdevice comprises a nucleic acids binding region and a handle region, and the 3D-printed microdevice is composed of more than two monomer 3D-printed microdevices through a joining region; and the nucleic acids binding region is made of photosensitive resin or thermoplastic. An extraction method for the nucleic acids extraction system based on a 3D-printed microdevice is used to bind, clean and elute nucleic acids by moving the monomer 3D-printed microdevice or the 3D-printed microdevice among a solution containing target nucleic acids, a washing buffer and an elution buffer.

### TECHNICAL FIELD

The present invention belongs to the technical field of nucleic acids extraction, and particularly relates to a nucleic acids extraction system and method based on a 3D-printed microdevice.

### BACKGROUND

Genes are the main functional units of heredity, and contain specific sequences of nucleic acid bases that can encode most of the proteins required for the functions of organisms. As a gene carrier, the nucleic acid-based in vitro molecular detection technology has become a powerful tool for biological research. To extract nucleic acids from organisms, it is necessary to separate and purify nucleic acids from complex systems containing various biomacromolecules such as protein, polysaccharide and fat, which is a prerequisite for biological analysis such as PCR and sequencing. The quality of nucleic acids extraction has significant effect on the accuracy and sensitivity of subsequent analysis. Inhibitory components (heme and globulin) remaining in biological samples and organic matters and salts remaining in the separation and purification processes are important factors that affect the sensitivity and analysis robustness of PCR.

The traditional nucleic acids separation methods are liquid phase separation based on organic solvent extraction, such as Chomczynski's method, alkaline lysis, phenol chloride extraction and ethidium bromide-calcium chloride gradient centrifugation. The liquid phase separation methods are effective, but have significant limitations: a large quantity of required samples, long separation time, easy pollution and high degradation rate. In addition, the residue of organic solvents in the liquid phase separation methods significantly increases the risk of PCR inhibitor residues. In order to improve the purity and concentration of extracted nucleic acid samples, the nucleic acids extraction methods are constantly updated, and the solid phase nucleic acids extraction method is favored by more scholars at present. In the solid phase nucleic acids extraction method, polymer materials with binding effect on nucleic acids are mainly used to bind nucleic acids under certain conditions and then carry out desorption under appropriate conditions to realize separation of nucleic acids. Compared with the liquid phase extraction methods, the solid phase extraction methods reduce the use of organic solvents and have simple operation and short separation time, samples are not easy to pollute and degrade in the experimental process, and the extracted nucleic acids are relatively high in both concentration and purity.

The solid phase nucleic acids separation methods are mainly based on polymer materials that can adsorb nucleic acids. With the development of magnetic materials, magnetic separation with magnetic particles as nucleic acids binding carriers has become the main means of solid phase nucleic acids separation due to the characteristic of capability of quickly moving in an external magnetic field after binding with nucleic acids. Magnetic separation avoids complicated centrifugation operation in the traditional nucleic acids separation process, and commercially available magnetic separation usually takes about only 15 min (which is reported to be 14.5 min in literature: Zou Y, Mason M G, Wang Y, Wee E, Turni C, Blackall P J, et al. (2017) Nucleic acid purification from plants, animals and microbes in under 30 seconds. PLoS Biol 15(11): e2003916.) and has high throughput in the downstream and automation application prospect, but magnetic separation is difficult to avoid multiple pipetting operations, needs multiple pipettes and is easy to produce errors in manual operation. Meanwhile, it is reported that magnetic separation has common problems of difficulty in nucleic acids desorption of particle materials and low efficiency of DNA extraction, which limits development and application of magnetic separation. Although it is reported that the problems can be avoided by using magnetic nanometer particles and DNA complexes directly as PCR templates, the magnetic particles strongly inhibit the PCR amplification process, and an elution process is inevitable. In addition, the immanent phenomena of agglomeration and sedimentation of magnetic nanometer particles affect the stability of the separation process to a large extent in high-throughput parallel batch processing of samples. In magnetic separation, due to the sedimentation of nanometer particles, nucleic acids and magnetic nanometer particles cannot bind fully, which affects the extraction yield, because the agglomeration of nanometer particles may cause incomplete cleaning and impurity wrapping, affecting the extraction quality of magnetic separation.

Compared with magnetic separation, solid phase nucleic acids separation based on non-magnetic materials is developed slowly. In the current non-magnetic solid phase separation, non-magnetic solid phase materials bound with nucleic acids can be separated from a lysate only by complicated operations such as filtration, centrifugation and pipetting. Compared with magnetic separation which can move quickly and separate nucleic acids by an external magnetic field, non-magnetic solid phase separation often requires more and more intensive manual operations, the efficiency and the throughput are relatively low, and downstream application is hindered, which are key bottlenecks restricting development and application of non-magnetic solid phase separation.

The 3D printing technologies, as the interface material construction technologies developed rapidly in recent years, can achieve the goals of flexible structure design and excellent molding effect by virtue of characteristic of high processing accuracy at the submillimeter and micron scales.

### SUMMARY

Aiming at the bottleneck problems of dependence on complicated operations such as filtration, centrifugation and pipetting, low throughput and difficulty in realizing automation commonly existing in non-magnetic solid phase separation, the present invention provides a nucleic acids extraction system and method based on a 3D-printed microdevice by taking the advantage of high processing accuracy of 3D printing.

The technical solution of the present invention is as follows:

A nucleic acids extraction system based on a 3D-printed microdevice, is a monomer 3D-printed microdevice or a 3D-printed microdevice prepared by 3D printing technologies; and the monomer 3D-printed microdevice comprises a nucleic acids binding region and a handle region, the 3D-printed microdevice is composed of more than two monomer 3D-printed microdevices and a joining region used for connecting monomer 3D-printed microdevices, and the handle region ends of the monomer 3D-printed microdevices, which are far away from the nucleic acids binding region, are connected in parallel to the joining region. Preferably, the tops of handles of 8 monomer 3D-printed microdevices are connected in parallel through the joining region, forming a special-shaped 3D-printed microdevice used in conjunction with a 8-tube strip, as shown in FIG. 3; or further, the handle region ends of 96 or 384 monomer 3D-printed microdevices are connected to and distributed on the joining region for use in conjunction with a 96-well plate and a 384-well plate.

The nucleic acids binding region of the monomer 3D-printed microdevice comprises type a, type b, type c, type d, type e and type f, wherein the type a is in a shape of cone, and the center position of the bottom surface of the cone is combined with the handle region; the type b is in a shape of conoid, the vertex of the cone is smoothed, and the center position of the bottom surface of the cone is combined with the handle region; the type c is in a shape of cylinder and hemispheroid, the diameter of the cylinder is the same as that of the hemispheroid, the center position of the bottom surface of one end of the cylinder is combined with the handle region, and the bottom surface of the other end is combined with the maximum diameter surface of the hemispheroid; the type d is in a shape of hemispheroid, and the center position of the maximum diameter surface of the hemispheroid is combined with the handle region; the type e is in a shape of cylinder and cone, the diameter of the cylinder is the same as that of the bottom surface of the cone, the center position of one end of the cylinder is combined with the handle region, and the other end is combined with the bottom surface of the cone; and the type f is in a shape of cylinder and conoid, the diameter of the cylinder is the same as that of the bottom surface of the conoid, the center position of one end of the cylinder is combined with the handle region, the other end is combined with the bottom surface of the conoid, and the vertex of the conoid is smoothed.

The structure of the monomer 3D-printed microdevice or the 3D-printed microdevice is designed, drawn and set through software, preferably, through 3D MAX software.

Further, in the above technical solution, the 3D printing technologies comprise: fused deposition modeling (FDM), stereo lithography apparatus (SLA), digital light processing (DLP) and selective laser sintering (SLS).

Further, in the above technical solution, the nucleic acids binding region has a smooth or rough surface, with or without a microstructure; the microstructure comprises one or a combination of more than two of thread structure, groove structure, porous structure, porous channel structure, coarse grain and convex structure; the number of the thread structure or groove structure may be zero or at least one; the coarse grain may be in any shape; the porous structure, the porous channel structure and the convex structure are in nanometer size or micron size; the groove structure and the convex structure may be in any shape, comprising cylinder, cone, platform and spheroid or in an irregular shape; and the microstructure can be distributed in any position of the nucleic acids binding region, and can be combined and distributed in any way when the number thereof is more than one.

Further, in the above technical solution, the handle region is in a shape of cylinder or prism and is made of photosensitive resin or thermoplastic, the nucleic acids binding region is made of photosensitive resin or thermoplastic, and the material of the handle region is the same as or different from that of the nucleic acids binding region; the joining region may be made of any material and have any structure, and when the joining region is completed through 3D printing technologies, the material of the joining region is photosensitive resin or thermoplastic and is the same as or different from that of the nucleic acids binding region and the handle region; and when the joining region is independent of 3D printing, the joining region has a structure with a certain rigidity where a plurality of monomer 3D-printed microdevices can be fixed and distributed.

The joining region, the handle region and the nucleic acids binding region are prepared integrally or separately through 3D printing technologies, and assembled by bonding or buckling when being prepared separately.

Further, in the above technical solution, the photosensitive resin is polyacrylic acid (PAA), polyethylene glycol diacrylate (PEGDA) or polycarbonate.

Further, in the above technical solution, the nucleic acids binding region is loaded or not loaded with functional groups or particle materials, wherein the functional groups comprise amino groups, carboxyl groups and hydroxyl groups; the particle materials comprise inorganic particle materials or metallic particle materials, and the inorganic particle materials comprise silicon dioxide, titanium dioxide, manganese dioxide, ferroferric oxide, graphene oxide, mica, etc.; and the metallic particle materials comprise gold, silver, iron, etc. When the nucleic acids binding region is loaded with functional groups or particle materials, a compound with the functional groups or the particle materials are mixed in the material of the nucleic acids binding region, and then the monomer 3D-printed microdevice or the 3D-printed microdevice is prepared through 3D printing technologies; or particles are loaded or surface functional groups are modified after the monomer 3D-printed microdevice or the 3D-printed microdevice is prepared.

Further, in the above technical solution, the nucleic acids extraction system based on a 3D-printed microdevice is used in conjunction with a centrifugal tube. When the nucleic acids extraction system based on a 3D-printed microdevice is placed in an EP tube, the height of the part of the handle region exposed from the centrifugal tube is not less than 3 mm; and preferably, the size of a microdevice used in conjunction with a single 0.2-2 mL centrifugal tube is as follows: a handle b has the height of 5-40 mm, the cylinder has the diameter of 2-5 mm, and the cuboid has the length of 2-5 mm and the width of 1-5 mm. The nucleic acids binding region has the size changing with the size of the matched target centrifugal tube, and can be placed at the bottom of the centrifuge tube; preferably, for a microdevice used in conjunction with a 0.2 mL centrifugal tube, the size of the nucleic acids binding region is: 2-5 mm (width)×5-20 mm (height); for a microdevice used in conjunction with a 0.5 mL centrifugal tube, the size of the nucleic acids binding region is: 2-6 mm (width)×5-26 mm (height); for a microdevice used in conjunction with a 1.5/2 mL centrifugal tube, the size of the nucleic acids binding region is: 3-9 mm (width)×5-35 mm (height); and the bottom of the nucleic acids binding region is a hemispherical cylinder, preferably, for a microdevice used in conjunction with a 1.5/2 mL centrifugal tube, the hemispherical part has the height of 2-4 mm and the diameter of 7-8 mm, and the cylinder part has the diameter of 7-8 mm and the height of 0-10 mm.

The present invention also provides a nucleic acids extraction method for a nucleic acids extraction system based on a 3D-printed microdevice, comprising the following steps:

(1) Inserting the nucleic acids binding region of a monomer 3D-printed microdevice or a 3D-printed microdevice into a solution containing target nucleic acids for nucleic acids binding;

(2) Moving the handle region or the joining region of the monomer 3D-printed microdevice or the 3D-printed microdevice completing step (1) by hand or a machine to make the nucleic acids binding region inserted into a washing buffer for nucleic acids cleaning;

(3) Taking out and drying the monomer 3D-printed microdevice or the 3D-printed microdevice completing step (2);

(4) Moving the handle region or the joining region of the monomer 3D-printed microdevice or the 3D-printed microdevice completing step (3) by hand or a machine to make the nucleic acids binding region placed into an elution buffer for nucleic acid elution, and the obtained elution buffer is the target nucleic acids extraction solution.

Further, in the above technical solution, in step (1), the monomer 3D-printed microdevice or the 3D-printed microdevice is placed into the solution containing target nucleic acids, wherein the target nucleic acids can be one or a mixture of more than two of RNA, genomic DNA and plasmid DNA; and the solution containing target nucleic acids may be any solution with a single component or multiple components from any source, a system derived from the lysis of biological samples with a lysate, or a mixed solution or single component solution containing the target nucleic acids.

The lysate refers to a buffer capable of releasing nucleic acids from a sample into a solution, and comprises CTAB lysate, NaHCO₃ lysate, Chelex lysate, proteinase K lysate, sodium dodecyl sulfate (SDS) lysate or Trizol lysate. The CTAB lysate comprises: 1 wt %-3 wt % of CTAB, 0.5-5 M of NaCl, 0.01-0.05 M of EDTA, 0.05-0.5 M of Tris-HCl and 0.05%-0.5% of mercaptoethanol, the NaHCO₃ lysate comprises: 0.05-1.00 M of NaHCO₃ and 0.5%-10% of SDS, the Chelex lysate comprises: 0.5%-20% of Chelex-100 and 0.2-5 M of DTT, and the proteinase K lysate comprises: 20-500 mM Tris-HCl, 10-50 mM EDTA, 100-1000 mM NaCl, 0.1%-10% of SDS and 5-30 μg/mL proteinase K; the SDS lysate comprises 0.5 wt %-20 wt % of SDS, wherein proteinase K is added at 0-30 μg/mL; and the Trizol lysate is a commercially available or self-prepared solution containing Trizol. The lysate is added or not added with RNA digestive enzyme or DNA digestive enzyme, and the lysis comprises oscillation and blending. The lysis time is 1 min-24 h, and the lysis temperature is −20-100° C.

The biological samples comprise animal samples or plant samples, including blood, animal and plant tissues, or mixtures; the blood comprises liquid whole blood, blood cell solution and dried blood spot samples; the animal tissues may be any part and any organ of animals; and the plant tissues comprise any component of any part of plants.

The specific steps of nucleic acids binding in step (1) are as follows: inserting the binding region of a monomer 3D-printed microdevice or a 3D-printed microdevice into a mixed solution containing target nucleic acids, and adding or not adding an auxiliary binding solvent, wherein the auxiliary binding solvent comprises one or a mixed solution of isopropyl alcohol and absolute ethyl alcohol, and the volume of the auxiliary binding solvent is 0.6-0.8 time that of the lysate; and the binding time is 5 s-24 h;

The nucleic acids cleaning in step (2) is carried out 1-5 times; and the cleaning time is 2 s-1 min each time;

The drying in step (3) can be carried out at room temperature or under the heating condition, and the drying time is 1 min-24 h;

The elution buffer in step (4) is a solution capable of separating nucleic acids bound to the monomer 3D-printed microdevice or the 3D-printed microdevice, comprising water, PBS buffer, TE buffer and downstream PCR reaction liquid; and the elution time is 5 s-5 min.

### Beneficial Effects of Invention

1. The present invention discloses a system and method for nucleic acids extraction using a 3D-printed microdevice for the first time, which is a non-magnetic solid phase nucleic acids extraction technology. In solid phase nucleic acids separation technology, magnetic separation is generally considered to be fast and have high throughput, usually taking about only 15 min. The nucleic acids extraction method of the present invention can obtain higher separation speed, simpler operation process and more stable separation effect than magnetic separation. Nucleic acids binding and cleaning can be completed within 30 s by using the monomer 3D-printed microdevice and the 3D-printed microdevice for nucleic acids separation, without the need of pipetting, centrifugation, filtration and other operations; meanwhile, with the monomer 3D-printed microdevice or the 3D-printed microdevice, the problems of insufficient nucleic acids binding and impurity wrapping caused by sedimentation and aggregation of magnetic bead particles do not exit, and the stability and the quality of nucleic acids extraction are effectively guaranteed; and the present invention breaks through the bottleneck problem in the current solid phase nucleic acids extraction, which is one of the important innovation points of the present invention.

2. The nucleic acids extraction system based on a 3D-printed microdevice disclosed by the present invention has the advantages of high throughput and high efficiency. When comprising 8, 96 and even 384 monomer 3D-printed microdevices used in conjunction with a 0.2 mL centrifugal tube, the 3D-printed microdevice can be used in conjunction with a 8-tube strip, a 96-well plate and a 384-well plate through the connection and arrangement of the joining region to parallelly complete separation of a plurality of nucleic acid samples, which significantly improves extraction throughput and efficiency and is another innovation point of the present invention.

3. The nucleic acids extraction system and method based on a 3D-printed microdevice disclosed by the present invention has automation application prospect. The method of the present invention can realize automatic nucleic acids extraction by designing automation equipment instead of manual operation and moving the microdevice among different solutions by a machine independently of complicated operations such as centrifugation and pipetting, which is another innovation point of the present invention.

4. The nucleic acids extraction system and method based on a 3D-printed microdevice disclosed by the present invention has high adjustment flexibility. First, microdevices with different sizes and different shapes can be obtained by setting parameters according to different requirements of downstream biomolecular technology application, and are used in conjunction with centrifugal tubes of different types and specifications, so as to flexibly meet application requirements; and second, the nucleic acids separation efficiency and selectivity of the monomer 3D-printed microdevice or the 3D-printed microdevice can be adjusted by adjusting the structure design, the functional group modification and the particle loading of the nucleic acid bindings region of the microdevice.

5. The nucleic acids extraction system and method based on a 3D-printed microdevice disclosed by the present invention has low cost and high use flexibility. 3D printing raw materials are cheap and easy to obtain, and the printing process is convenient and fast, which can significantly reduce the economic cost; and the whole nucleic acids extraction process can be completed in an operating environment of 1 m², which saves space and does not rely on electrical appliance or pipettor, with low equipment requirements and high use flexibility.

### DESCRIPTION OF DRAWINGS

FIG. 1 is a structural schematic diagram of a preferred monomer 3D-printed microdevice of the present invention.

FIG. 2 is a structural schematic diagram of a monomer 3D-printed microdevice prepared in embodiment 1 of the present invention. FIG. 2(a) to FIG. 2(h) respectively show monomer 3D-printed microdevices 1-8.

FIG. 3 is a structural schematic diagram of a special-shaped 3D-printed microdevice of embodiment 4 of the present invention.

FIG. 4 is an agarose gel electrophoretogram of embodiment 4 of the present invention, wherein lane 1 is negative control, lanes 2-9 are 8 samples, and M is DL2000 marker.

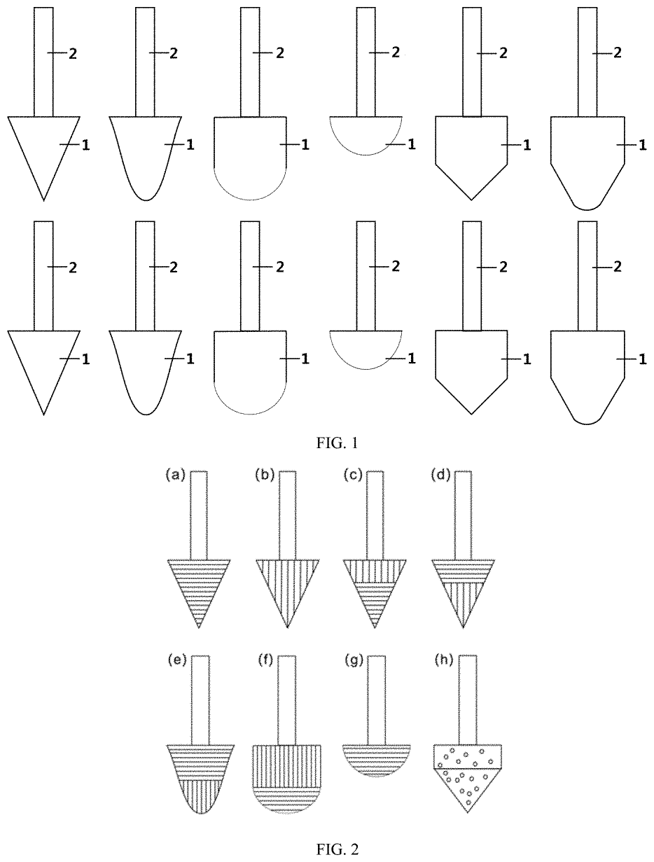

In the figures: 1 nucleic acids binding region; 2 handle region; and 3 joining region.

### DETAILED DESCRIPTION

The contents of the present invention are further described below in combination with the technical solution. It should be pointed out that the following descriptions are all illustrative and are intended to provide further description of the present invention. Unless otherwise specified, all scientific and technical terms used in the present invention have the same meanings as those generally understood by those skilled in the art of the present invention.

The materials and the actual and experimental equipment involved in embodiments of the present invention are in line with commercially available products in the related technical fields of chemical industry and biology.

Primers involved in the embodiments of the present invention are synthesized by a commissioned bioengineering company, and the specific primer information is as follows:

SEQ ID NO: 1:
5′-actgggataatacgatagaag-3′
SEQ ID NO: 2:
5′-gtgcgttaggattagttatgt-3′

### Embodiment 1

3D MAX software is adopted to design, draw and set the structure. Photosensitive resin, polyacrylic acid (PAA), as a raw material, is subjected to photocuring reaction by the DLP 3D printing technology at wavelength of 400-800 nm to prepare a monomer 3D-printed microdevice, and the structural schematic diagram is shown in FIG. 2(a) to FIG. 2(h), wherein the handle regions of monomer 3D-printed microdevices shown in FIG. 2(a) to FIG. 2(f) are cylindrical, and have diameters of 3 mm, 3 mm, 5 mm, 4 mm, 4 mm and 6 mm and heights of 1.5 cm, 3.0 cm, 12.0 cm, 2.0 cm, 4.0 cm and 12 cm; and the handle regions of monomer 3D-printed microdevices shown in FIG. 2(g) to FIG. 2(h) are respectively in a shape of square cylinder, the handle region of a monomer 3D-printed microdevice shown in FIG. 2(g) is 5 mm in length and width and 6.0 cm in height, and the handle region of the monomer 3D-printed microdevice shown in FIG. 2(h) is 6 mm in length and width and 12.0 cm in height. The nucleic acids binding regions of monomer 3D-printed microdevices shown in FIG. 2(a) to FIG. 2(h) have diameters of 0.38 cm, 0.4 cm, 1.2 cm, 0.6 cm, 0.6 cm, 2.0 cm, 1.0 cm and 2.0 cm and heights of 0.8 cm, 1.0 cm, 2.0 cm, 1.0 cm, 1.0 cm, 2.5, cm, 1.0 cm and 2.5 cm. The nucleic acids binding regions of monomer 3D-printed microdevices shown in FIG. 2(a), FIG. 2(c) and FIG. 2(d)-FIG. 2(g) respectively have a helical structure, which has 8 layers, 4 layers, 6 layers, 10 layers, 3 layers and 5 layers respectively; The nucleic acids binding regions of monomer 3D-printed microdevices shown in FIG. 2(b) to FIG. 2(f) respectively have a groove structure, which comprises 6 grooves, 8 grooves, 3 grooves, 4 grooves and 20 grooves; and the nucleic acids binding region of a monomer 3D-printed microdevice shown in FIG. 2(h) has a porous structure.

Results show that monomer 3D-printed microdevices shown in FIG. 2(a)-FIG. 2(h) can be placed into a 0.2 ml conical centrifuge tube, a 0.5 ml conical centrifuge tube, a 15 ml conical centrifuge tube, a 1.5 ml conical centrifuge tube, a 2.0 ml conical centrifuge tube, a 50 ml round-bottom centrifuge tube, a 5 ml round-bottom centrifuge tube, and a 50 ml conical centrifuge tube respectively.

In the embodiment, PAA is used as the raw material, which is easy to obtain, and the 3D printing process is simple and fast, which can significantly reduce the economic cost; and microdevices with different sizes and different shapes can be obtained by setting parameters according to different requirements of downstream biomolecular technology application, and are used in conjunction with centrifugal tubes of different types and specifications, so as to flexibly meet application requirements, with high adjustment flexibility.

### Embodiment 2

A part of silkworm chrysalis tissues are taken and placed in a 1.5 mL EP tube. 200 μL of proteinase K lysate (100 mmol of Tris-HCl, 25 mmol of EDTA, 500 mmol of NaCl and 1% of SDS) and 5 μL of proteinase K solution are added into the EP tube, oscillated and blended to lyse the extracted sample tissues, and placed at room temperature for 30 min, 150 μL of absolute ethyl alcohol is added, the monomer 3D-printed microdevice shown in FIG. 2(d) is placed into the EP tube to make the nucleic acids binding region of the monomer 3D-printed microdevice inserted into the solution, the handle region is held by hand or a machine, the 3D-printed microdevice is slightly shaken to blend the solution, the monomer 3D-printed microdevice is transferred to an EP tube with 200 μL of washing buffer after 10 s to make the nucleic acids binding region of the monomer 3D-printed microdevice inserted into the washing buffer, then the handle region is held by hand or a machine, and the monomer 3D-printed microdevice is slightly shaken for 5 s, and then transferred to the washing buffer repeatedly two times; and the monomer 3D-printed microdevice is taken out from the washing buffer, the residual droplets on the monomer 3D-printed microdevice are shaken off, the monomer 3D-printed microdevice is dried in a 37° C. blast incubator for 1 min, and the nucleic acids binding region of the monomer 3D-printed microdevice is placed into a 1.5 mL EP tube with 50 μL of elution buffer and slightly shaken for 30 s for elution.

3 μL of elution buffer is taken and tested with an ultraviolet spectrophotometer three times, and for the extracted nucleic acid samples, 260/230 is 1.610-1.728, 260/280 is 1.817-1.902, and the concentration is 42.32-48.274 ng/μL.

Results show that the DNA samples extracted by the nucleic acids extraction system based on a 3D-printed microdevice of the present invention from the lysate of animal tissue samples have better quality and almost no pollution of protein, salt, polysaccharide, etc.

The embodiment illustrates that when the nucleic acids extraction system based on a 3D-printed microdevice is used for nucleic acids extraction, nucleic acid binding and cleaning can be completed in 25 s without pipetting, centrifugation, filtration and other operations, which is convenient and rapid with good extraction quality.

### Embodiment 3

4.5 ml of Escherichia coli culture solution is taken and added into a 5 ml EP tube, and centrifuged to remove supernatant, 500 ml of sterile resuspension (50 mmol/L glucose, 25 mmol/L Tris with PH=8.0, and 10 mmol/L EDTA with PH=8.0) is added to suspend thalli, then 1 mL of lysate (0.2 M of NaOH and 1% of SDS) is added, the centrifugal tube is turned upside down five times for 2 min to blend the solution until the solution is thick but clear, then 750 μL of neutralization buffer (5 mol/L potassium acetate and 5 mol/L glacial acetic acid) is added, the centrifugal tube is immediately turned upside down several times until flocculent precipitation appears in the solution, the solution is centrifuged at 12000 r/min for 10 min, and 2 mL of supernatant is drawn carefully and transferred to another 5 mL centrifugal tube to obtain a solution containing plasmid DNA.

1500 μL of absolute ethyl alcohol is added to the solution containing plasmid DNA, the monomer 3D-printed microdevice shown in FIG. 2(g) is placed into the solution to make the nucleic acids binding region of the monomer 3D-printed microdevice inserted into the solution, the handle region is held by hand, the monomer 3D-printed microdevice is slightly shaken to blend the solution, and then transferred to an EP tube with 2000 μL of washing buffer (75% alcohol) after 25-30 s to make the nucleic acids binding region inserted into the washing buffer, then the handle region is held by hand, and the monomer 3D-printed microdevice is slightly shaken for 10-15 s, and then transferred to the washing buffer repeatedly 2-3 times; and the monomer 3D-printed microdevice is taken out from the washing buffer, the residual droplets on the monomer 3D-printed microdevice are shaken off, the monomer 3D-printed microdevice is dried at room temperature for 15 min, and the nucleic acids binding region of the monomer 3D-printed microdevice is placed into 100 μL of elution buffer and slightly shaken for 30 s for elution.

The extracted plasmid DNA is tested with an ultraviolet spectrophotometer three times, and for the plasmid DNA, 260/230 is 1.723-1.789, 260/280 is 1.857-1.931, and the concentration is 178.30-181.464 ng/μL.

Results show that the plasmid DNA samples extracted by the monomer 3D-printed microdevice of the present invention from bacteria have high quality and no pollution. The plasmid DNA separation process does not need pipetting, centrifugation, filtration and other operations, which is convenient and rapid with good extraction quality.

### Embodiment 4

(1) Preparation of Special-Shaped 3D-Printed Microdevice (Composed of Eight Monomer 3D-Printed Microdevices Connected by a Joining Region)

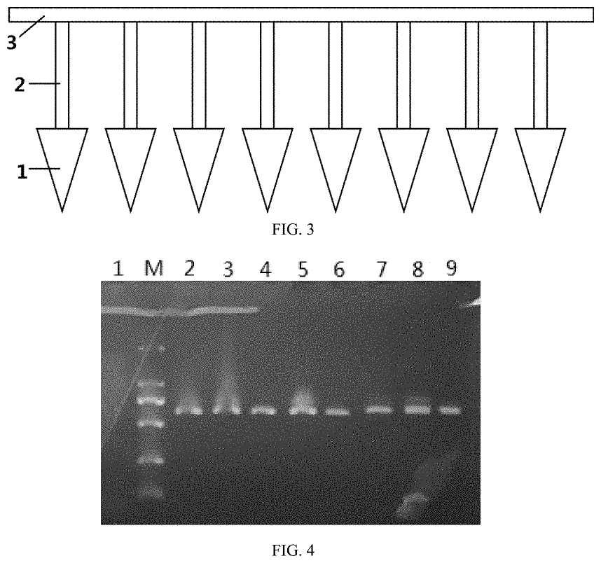

3D MAX software is adopted to design, draw and set the structure. Photosensitive resin (PAA), as a raw material, is subjected to photocuring reaction by the DLP 3D printing technology at wavelength of 400-800 nm to prepare a 3D-printed microdevice having a structure shown in FIG. 3, which has the nucleic acids binding region with the height of 9 mm and the diameter of 3.8 mm and the handle region with the height of 21 mm and can be placed in a 8-tube strip for use.

(2) Preparation of Target Nucleic Acid Solution

A frozen clam is taken, a tissue block weighing about 500 mg thereof is directly put into a mortar sterilized by high temperature and high pressure, added with liquid nitrogen and ground rapidly, after the tissue is softened, a small amount of liquid nitrogen is added, the tissue block is ground again, and the above operation is repeated three times. Then 150-200 mg of tissue samples is taken, added with 2 ml of Trizol and fully blended with an electric homogenizer for 1-2 min. Centrifugation is conducted at 12000 r/min for 5 min, precipitation is discarded, 400 μL of chloroform is added, the centrifugal tube is covered tightly, and the mixture is shaken by hand for 15 s and placed at room temperature for 10 min. Then, the mixture is centrifuged at 4° C. at 12000 g for 15 min, and the aqueous phase is transferred to a new EP tube to obtain a solution containing RNA.

(3) High Throughput and Rapid RNA Separation by Special-Shaped Microdevice

80 μL of RNA solution is taken and added into each reaction hole of a 8-tube strip, a special-shaped microdevice is placed into each reaction hole to make the nucleic acids binding region completely inserted into the solution, 55 μL of isopropyl alcohol is added, the special-shaped microdevice is slightly shaken to blend the solution, and after 5-10 s, transferred to an EP tube with 200 μL of washing buffer (75% alcohol), and the special-shaped microdevice is slightly shaken for 5-10 s, and then transferred to the washing buffer repeatedly one time; and the special-shaped microdevice is taken out from the washing buffer, the residual droplets on the special-shaped microdevice are shaken off, and the special-shaped microdevice is dried at room temperature for 5 min, placed into 40 μL of elution buffer and slightly shaken for 30 s for elution. The RNA concentration of each elution buffer is tested with an ultraviolet spectrophotometer between 41 ng/μL and 52 ng/μL, A260/230 is 1.703-1.920, and 260/280 is 1.974-2.133.

(4) Downstream Application of Extracted RNA

7 μL of liquid is taken from each reaction hole of the 8-tube strip, cDNA is prepared through a reverse transcription kit (Takara, Code No. RR047A) according to operating instructions, and 1 μL of product is respectively taken as an PCR amplification template to prepare PCR reaction liquid: every 25 μL of system contains 5 μL of primer (10 pM) shown in SEQ ID NO:1 and SEQ ID NO:2, 1 μL of template, 0.25 μL of ExTaq, 2.5 μL of 10×ExBuffer, 2 μL of dNTPs and 17.25 μL of deionized water, and sterile distilled water is used as a negative reference template. The PCR condition is 4 min at 95° C.; 35 cycles are performed at 95° C. for 30 s, 55° C. for 30 s and 72° C. for 60 s, and extension is performed at 72° C. for 5 min. The obtained PCR products are subjected to gel electrophoresis with 2% agarose, and the results are observed under UV light after the electrophoresis, as shown in FIG. 4.

Results show that FIG. 4 shows negative reference, marker and PCR amplification products of 8 samples from left to right, target strips appear in the 8 samples, and no target strip appears in the negative reference, indicating that 8 solutions used as templates contain target nucleic acids.

The embodiment shows that the RNA separated by the 3D-printed microdevice has good quality and less pollution and can fully meet the experimental requirements of downstream molecular technologies.

The embodiment also shows that the special-shaped microdevice is used for nucleic acids extraction, which can extract target nucleic acids at the same time with high throughput, effectively breaks through the bottleneck of low separation throughput of non-magnetic solid phase separation due to dependence on multi-step centrifugation, pipetting and other complicated operations and is conducive to realization of automation.

### Embodiment 5

Photosensitive resin (PAA), as a raw material, is subjected to photocuring reaction by the DLP 3D printing technology at wavelength of 400-800 nm to prepare a cubic strip-shaped microdevice: 2 mm wide, 1 mm thick and 4 cm high, and the cubic strip-shaped microdevice has no special microstructure design.

Animal tissues (shellfish tissues) are taken, lysate (100 mmol of Tris-HCl, 25 mmol of EDTA, 500 mmol of NaCl and 1% SDS) and 5 μL of proteinase K are added, after warm bath at 55° C. for 2 h, 100 μL is respectively taken and added into two 0.5 mL centrifugal tubes which are numbered 1 # and 2 #, and 10 μL of RNA digestive enzyme is respectively added into the two centrifugal tubes and placed at room temperature for 30 min to obtain an RNA-free solution containing target DNA.

70 μL of absolute ethyl alcohol is added into the 1 # tube, and the monomer 3D-printed microdevice shown in FIG. 2(b) and prepared in embodiment 1 is placed into the 1 # tube. 70 μL of absolute ethyl alcohol is added into the 2 # tube, and the cubic strip-shaped microdevice is placed into the 2 # tube. The microdevices are slightly shaken to blend the solutions, and after 15-20 s, respectively transferred to an EP tube with 100 μL of washing buffer (75% alcohol), and the microdevices are slightly shaken for 15-20 s, and then transferred to the washing buffers repeatedly three times; and the microdevices are respectively taken out from the washing buffers, the residual droplets on each microdevice are shaken off, and each microdevice is dried in a 37° C. blast incubator for 3 min, placed into 40 μL of elution buffer and slightly shaken for 30 s for elution.

The DNA concentration of the extracted nucleic acids is tested with an ultraviolet spectrophotometer, and for the nucleic acid sample extracted from the 1 # tube, 260/230 is 1.864, 260/280 is 1.902, and the concentration is 90.547 ng/μL. For the nucleic acid sample extracted from the 2 # tube, 260/230 is 1.616, 260/280 is 1.736, and the concentration is 21.233 ng/μL.

Results show that the embodiment can quickly and effectively separate DNA with high quality, and the separated and purified DNA has low pollution and high purity. Meanwhile, compared with other monomer 3D-printed microdevices, the monomer 3D-printed microdevice preferred in the embodiment, especially the monomer 3D-printed microdevice with microstructure, has higher nucleic acids separation ability.

The technology of the present invention is described through preferred embodiments. Related technicians can obviously modify or appropriately change and combine the system and method described herein without departing from the scope of the content and spirit of the present invention to realize the technology of the present invention. It should be pointed out that all similar replacements and changes, such as reasonable adjustment, change and combination of shape, size, surface and structure of monomer 3D-printed microdevice or 3D-printed microdevice, reasonable composition adjustment of lysate, washing buffer and elution buffer, reasonable extension and contraction of operation time, and reasonable change of operating temperature, are apparent to those skilled in the art and are considered to be included in the spirit, scope and content of the present invention.

1. A nucleic acids extraction system based on a 3D-printed microdevice, wherein the nucleic acids extraction system based on a 3D-printed microdevice is a monomer 3D-printed microdevice or a 3D-printed microdevice prepared by 3D printing technologies; the monomer 3D-printed microdevice comprises a nucleic acids binding region and a handle region, the 3D-printed microdevice is composed of more than two monomer 3D-printed microdevices and a joining region used for connecting monomer 3D-printed microdevices, and the handle region ends of the monomer 3D-printed microdevices, which are far away from the nucleic acids binding region, are connected in parallel to the joining region; the nucleic acids binding region of the monomer 3D-printed microdevice comprises type a, type b, type c, type d, type e and type f, wherein the type a is in a shape of cone, and the center position of the bottom surface of the cone is combined with the handle region; the type b is in a shape of conoid, the vertex of the cone is smoothed, and the center position of the bottom surface of the cone is combined with the handle region; the type c is in a shape of cylinder and hemispheroid, the diameter of the cylinder is the same as that of the hemispheroid, the center position of the bottom surface of one end of the cylinder is combined with the handle region, and the bottom surface of the other end is combined with the maximum diameter surface of the hemispheroid; the type d is in a shape of hemispheroid, and the center position of the maximum diameter surface of the hemispheroid is combined with the handle region; the type e is in a shape of cylinder and cone, the diameter of the cylinder is the same as that of the bottom surface of the cone, the center position of one end of the cylinder is combined with the handle region, and the other end is combined with the bottom surface of the cone; and the type f is in a shape of cylinder and conoid, the diameter of the cylinder is the same as that of the bottom surface of the conoid, the center position of one end of the cylinder is combined with the handle region, the other end is combined with the bottom surface of the conoid, and the vertex of the conoid is smoothed.

2. The nucleic acids extraction system based on a 3D-printed microdevice according to claim 1, wherein the nucleic acids binding region has a smooth or rough surface, with or without a microstructure; the microstructure comprises one or a combination of more than two of thread structure, groove structure, porous structure, porous channel structure, coarse grain and convex structure; the number of the thread structure or groove structure is zero or at least one; the coarse grain is in any shape; the porous structure, the porous channel structure and the convex structure are in nanometer size or micron size; the groove structure and the convex structure may be in a shape of cylinder, cone, platform and spheroid or in an irregular shape; and the microstructure is distributed in any position of the nucleic acids binding region, and is combined and distributed in any way when the number thereof is more than one.

3. The nucleic acids extraction system based on a 3D-printed microdevice according to claim 1, wherein the handle region is in a shape of cylinder or prism; the handle region is made of photosensitive resin or thermoplastic, the nucleic acids binding region is made of photosensitive resin or thermoplastic, and the material of the handle region is the same as or different from that of the nucleic acids binding region; and the joining region, the handle region and the nucleic acids binding region are prepared integrally or separately through 3D printing technologies, and assembled by bonding or buckling when being prepared separately.

4. The nucleic acids extraction system based on a 3D-printed microdevice according to claim 1, wherein the nucleic acids binding region is loaded or not loaded with functional groups or particle materials, wherein the functional groups comprise amino groups, carboxyl groups and hydroxyl groups; the particle materials comprise inorganic particle materials or metallic particle materials, and the inorganic particle materials comprise silicon dioxide, titanium dioxide, manganese dioxide, ferroferric oxide, graphene oxide and mica; and the metallic particle materials comprise gold, silver and iron.

5. The nucleic acids extraction system based on a 3D-printed microdevice according to claim 1, wherein the nucleic acids extraction system based on a 3D-printed microdevice is used in conjunction with a centrifugal tube, and when the nucleic acids extraction system based on a 3D-printed microdevice is placed in a centrifugal tube, the height of the part of the handle region exposed from the centrifugal tube is not less than 3 mm.

6. A nucleic acids extraction method for the nucleic acids extraction system based on a 3D-printed microdevice according to claim 1, comprising the following steps:
(1) inserting the nucleic acids binding region of a monomer 3D-printed microdevice or a 3D-printed microdevice into a solution containing target nucleic acids for nucleic acids binding;
(2) moving the handle region or the joining region of the monomer 3D-printed microdevice or the 3D-printed microdevice completing step (1) by hand or a machine to make the nucleic acids binding region inserted into a washing buffer for nucleic acids cleaning;
(3) taking out and drying the monomer 3D-printed microdevice or the 3D-printed microdevice completing step (2);
(4) moving the handle region or the joining region of the monomer 3D-printed microdevice or the 3D-printed microdevice completing step (3) by hand or a machine to make the nucleic acids binding region placed into an elution buffer for nucleic acid elution, and the obtained elution buffer is the target nucleic acids extraction solution.

7. The nucleic acids extraction method according to claim 6, wherein
the specific steps of nucleic acids binding in step (1) are as follows: inserting the nucleic acids binding region of a monomer 3D-printed microdevice or a 3D-printed microdevice into a mixed solution containing target nucleic acids, and adding or not adding an auxiliary binding solvent, wherein the auxiliary binding solvent comprises one or a mixed solution of isopropyl alcohol and absolute ethyl alcohol, and the volume of the auxiliary binding solvent is 0.6-0.8 time that of the lysate; and the binding time is 5 s-24 h;
the nucleic acids cleaning in step (2) is carried out for 1-5 times; and the cleaning time is 2 s-1 min each time;
the drying in step (3) can be carried out at room temperature or under the heating condition, and the drying time is 1 min-24 h;
the elution buffer in step (4) is a solution capable of separating nucleic acids bound to the monomer 3D-printed microdevice or the 3D-printed microdevice, comprising water, PBS buffer, TE buffer and downstream PCR reaction liquid; and the elution time is 5 s-5 min.

8. The nucleic acids extraction method according to claim 6, wherein the target nucleic acids in step (1) comprise one or a mixture of more than two of RNA, genomic DNA and plasmid DNA.

9. The nucleic acids extraction method according to claim 6, wherein the lysate is CTAB lysate, NaHCO₃ lysate, Chelex lysate, proteinase K lysate, SDS lysate or Trizol lysate; the lysate is added or not added with RNA digestive enzyme or DNA digestive enzyme; and the lysis time is 1 min-24 h, and the lysis temperature is −20-100° C.

10. The nucleic acids extraction method according to claim 9, wherein the SDS lysate comprises 0.5 wt %-20 wt % of SDS, wherein proteinase K is added at 0-30 μg/mL.
